(19) United States
(12) Patent Application Publication
(10) Pub. No.: US 2025/0367444 A1
(43) Pub. Date: Dec. 4, 2025

(54) SYSTEM AND METHOD FOR OPTIMIZING RADIOFREQUENCY POWER DELIVERY BASED ON DIRECTION OF ELECTRIC FIELD

(71) Applicant: Korea University Research and Business Foundation, Seoul (KR)

(72) Inventors: Myong Geun YOON, Gapyeong-gun (KR); Geon O, Seoul (KR); Go Eun JUNG, Daejeon (KR)

(21) Appl. No.: 19/211,569

(22) Filed: May 19, 2025

(30) Foreign Application Priority Data
May 31, 2024 (KR) 10-2024-0071689

(51) Int. Cl. A61N 1/36; A61N 1/04; A61N 1/06

(52) U.S. Cl. CPC A61N 1/36002; A61N 1/0476; A61N 1/06; A61N 1/36034

(57) ABSTRACT

The present application relates to a system and method configured to maximize cell apoptosis and thereby optimize the effect of high-frequency electric field application. The electric field direction-based high-frequency power application optimization system includes a high-frequency power application system composed of a plurality of electrode pairs capable of applying electric fields in two or more directions to a region of interest (ROI) in a target subject. The system may include: an electrode pair-specific power application time setting unit for determining the duration of power application for each electrode pair, an electrode pair-specific power intensity setting unit for determining the power intensity to be applied to each electrode pair, and a power application sequence setting unit for determining the sequence in which power is applied to the respective electrode pairs.

### CROSS-REFERENCE TO RELATED APPLICATIONS

This application claims priority to and the benefit of Korean Patent Application No. 10-2024-0071689 filed in the Korean Intellectual Property Office on May 31, 2024,the entire contents of which are incorporated herein by reference.

### TECHNICAL FIELD

The present invention relates to a system and method for optimizing high-frequency power application based on the direction of the electric field. More specifically, the invention provides a technique for maximizing cell apoptosis and optimizing the effect of high-frequency application by using, as parameters, the total number of electrode pairs used for power application to a target object, the power application time for each electrode pair, the position and orientation of each electrode pair, the intensity of power applied to each electrode pair, and the sequence of power application among the electrode pairs.

### BACKGROUND ART

In the early 2000s, Professor Yoram Palti, a biophysicist in Israel, first discovered that the application of high-frequency electric fields ranging from 100 to 300 kHz to dividing cancer cells delays or inhibits their mitosis. In 2004, he published the world's first research findings on the therapeutic effects of high-frequency electric fields for cancer treatment in the journal Cancer Research. Since then, various studies have been conducted on electric field-based cancer therapy, which has gained significant attention in the oncology community due to its three key advantages.

The first advantage is that electric fields primarily affect dividing cells, thereby selectively targeting cancer cells that divide faster than normal cells. Consequently, the treatment is expected to result in significantly fewer side effects compared to conventional therapies. Indeed, according to publicly available data published in 2013, among nine side effect categories compared between chemotherapy and electric field therapy, electric field therapy exhibited substantially fewer side effects in seven categories and showed comparable levels in the remaining two.

The second advantage is that, despite being in the early stages of development, electric field-based cancer therapy has demonstrated superior therapeutic efficacy compared to conventional treatments. For example, in patients with glioblastoma multiforme (GBM), a highly malignant brain tumor, chemotherapy alone resulted in a progression-free survival (PFS) of 4.0 months, an overall survival (OS) of 16.0 months, and a two-year survival rate of 31%. However, when electric field therapy was added, the figures improved to 6.7 months for PFS, 20.9 months for OS, and a 43% two-year survival rate, representing approximately 1.7-, 1.3-, and 1.4-fold improvements, respectively.

The third advantage lies in the potential of electric fields to effectively target micro-tumors that are not visible on medical imaging modalities such as CT scans. When electric fields are applied broadly across the treatment region, not only the tumor but also surrounding tissues are exposed to meaningful field strength. This allows the inhibition of cancer cell proliferation in micro-tumors that may exist in the vicinity of the primary tumor but are too small to be visually detected, thereby potentially reducing the risk of metastasis significantly.

Electric field-based cancer therapy has received regulatory approval in several regions. In the United States, the FDA approved the treatment for recurrent GBM in 2011 and for newly diagnosed GBM in 2015. In Europe, the therapy has obtained CE marking and is currently practiced in approximately 2,000 hospitals across the United States, Germany, Switzerland, and other countries. Japan has also approved its use for recurrent GBM patients. The number of treated patients has increased rapidly—from 152 in 2014 to 8,813 in 2018—demonstrating more than a 50-fold growth.

The primary mechanism believed to be responsible for inhibiting cancer cell division during electric field therapy is dielectrophoresis. Dielectrophoresis refers to the force experienced by particles exposed to a non-uniform electric field, depending on the voltage and frequency of the field, as well as the permittivity and conductivity of the surrounding medium. In dividing cancer cells, a cleavage furrow forms between the two daughter cells. When this furrow is exposed to a non-uniform electric field, a field gradient is created, and a corresponding dielectrophoretic force increases. This force acts on polar biomolecules, such as tubulin, inside the cell, ultimately disrupting the mitotic process and inhibiting cell division.

According to current research, the magnitude of this dielectrophoretic force depends on the angle between the direction of the applied electric field and the cell's mitotic axis, as well as on the strength of the electric field. Specifically, the force is maximized when the electric field is aligned parallel (i.e., at 0° or) 180° to the mitotic axis and minimized when it is perpendicular (i.e., at 90°.

However, since the orientation of mitotic axes in tumor cells is randomly distributed, applying an alternating electric field in a single direction affects only those cancer cells whose mitotic axes are nearly aligned with that direction. For cells with mitotic axes nearly perpendicular to the field, the dielectrophoretic effect is negligible. To maximize the inhibition of cancer cell division across a broader population of tumor cells, it is more effective to apply alternating electric fields in multiple directions rather than a single fixed direction. In current clinical practice, this is commonly implemented by using two pairs of electrode arrays and alternating their directions periodically, applying equal treatment times in each direction.

Nonetheless, such a treatment strategy does not account for individual patient variability, such as anatomical structure, tumor location, the required number of field directions, the strength of the electric field in each direction, or the time-dependent effects of each directional application. Therefore, it is difficult to consider this method as an optimized, patient-specific treatment approach.

To achieve maximal inhibition of cancer cell proliferation via electric field therapy, high-frequency power must be applied in a manner optimized to the individual subject. This requires consideration of the subject's anatomical structure, the size, shape, and location of the region of interest (ROI), the total number of electric field directions to be applied, the orientation (or angle) of each field, the intensity of each directional field, and the application duration for each direction.

### SUMMARY OF THE INVENTION

Accordingly, in the relevant technical field, there is a need for an optimized scheme for maximizing the effect of high-frequency power application by utilizing, as parameters, the total number of electric field directions (n) applied to the region of interest (ROI) of the subject, the direction (θ) of each electric field, the electric field intensity (E) in each direction, and the application time (t) for each direction, in order to maximize the cell proliferation inhibition effect resulting from dielectrophoretic forces.

According to one embodiment, the present invention provides a high-frequency power application optimization system comprising a plurality of electrode pairs (110) capable of applying electric fields in two or more directions to a region of interest (ROI) within a three-dimensional subject. The system includes:
an electrode pair-specific power application time setting unit (102) configured to determine the power application time for each electrode pair (110),
an electrode pair-specific power intensity setting unit (104) configured to determine the intensity of power applied to each electrode pair (110), and
a power application sequence setting unit (106) configured to determine the sequence in which power is applied to each electrode pair (110),
   wherein the total number of electrode pairs used for power application, the position of each electrode pair, the power intensity applied to each electrode pair, the power application time for each electrode pair, and the sequence of power application are configured so as to maximize inhibition of cell division in the region of interest (ROI). In this system, the power application time of each electrode pair (100) is set such that power is applied sequentially without overlap among the electrode pairs, in a repeated manner. As a basic configuration, the power application time for each electrode pair (100) is set to be equal and applied in sequential cycles.

Each electrode forming the electrode pair (100) may consist of one or more individual electrodes. Furthermore, each electrode may be configured as an electrode array composed of multiple individual electrodes. In some embodiments, a portion of the individual electrodes comprising one electrode pair (110) may be reused as part of another electrode pair (110).

In the basic configuration, the positions of the electrode pairs (100) are arranged such that the angular intervals between adjacent electrode pairs are uniform. The system may further include a switching unit (108) capable of selecting each electrode pair (110) to apply power sequentially.

The high-frequency power applied is controlled to apply an alternating voltage in the range of 10 to 1000 kHz to the three-dimensional subject.

In another embodiment, the present invention provides a method for optimizing high-frequency power application using the aforementioned system to apply electric fields in two or more directions to a region of interest in a three-dimensional subject. The method includes:
a step of setting the total number of electrode pairs (110) used for applying power to the three-dimensional subject;
a step of setting the position or orientation of the electrode pairs (110) used for power application;
a step of setting the power intensity applied to each electrode pair (110) via the electrode pair-specific power intensity setting unit (104);
a step of setting the power application time for each electrode pair (110) via the electrode pair-specific power application time setting unit (102);
a step of setting the sequence of power application for each electrode pair (110) via the power application sequence setting unit (106); and
a step of sequentially applying power to the three-dimensional subject using the respective electrode pairs (110),
   wherein the total number of electrode pairs used, the position of each electrode pair, the intensity of the power applied, the application time, and the application sequence are configured so as to maximize inhibition of cell division in the region of interest (ROI).

According to an embodiment of the present invention, the effect of cell death induced by high-frequency power application can be optimized by maximizing apoptosis. This is achieved by using, as parameters, the total number of electric field directions applied to the region of interest, the direction of each electric field, the electric field intensity in each direction, and the application time for each direction.

### BRIEF DESCRIPTION OF THE DRAWINGS

FIG. 1 is a diagram illustrating the distribution of electric field intensity applied within a tumor cell according to the state of cell division and the frequency of an applied alternating electric field.

FIGS. 2(a), 2(b), and 2(c) are diagrams showing variations in the magnitude of the electric field formed inside the cell depending on the angle of the cell's mitotic axis.

FIGS. 3(a), 3(b), and 3(c) are diagrams showing variations in the dielectrophoretic force measured inside the cell according to the orientation of the cell's mitotic axis.

FIGS. 4(a), and 4(b) are diagrams illustrating changes in the magnitude of dielectrophoretic force measured inside the cell when the total number of applied electric field directions increases from one to four.

FIG. 5 is a graph showing the average magnitude of dielectrophoretic force from 0°0 to 180° for each graph in FIG. 4(b), represented as relative values when the magnitude in the single-direction case is normalized to 1.

FIGS. 6(a), and 6(b) are diagrams showing changes in dielectrophoretic force measured inside the cell when the application time per direction is varied in two-directional electric field applications.

FIG. 7 is a graph showing the average dielectrophoretic force from 0° to 180° for each graph in FIG. 6(b), expressed as relative values when the force in the horizontal-only 4-hour application is normalized to 1.

FIGS. 8(a), and 8(b) are diagrams showing changes in dielectrophoretic force measured inside the cell when the angle between the two electric field directions is varied.

FIG. 9 is a graph showing the average magnitude of dielectrophoretic force from 0° to 180° for each graph in FIG. 8(b), represented as relative values when the angle between directions is set to 30° and normalized to 1.

FIGS. 10(a), and 10(b) are graphs showing the average cell viability across various cell lines, comparing experimental groups to control groups depending on the intensity and duration of the applied external electric field.

FIGS. 11(a), and 11(b) are diagrams showing the direction of the electric fields and the tumor location within a human phantom model when electric fields are applied in two directions.

FIGS. 12 a), and 12(b) are graphs showing cell viability in experimental groups relative to control groups when the intensity and duration of electric field application vary by direction for the tumor shown in FIGS. 11(a) and 11(b).

FIG. 13 is a block diagram illustrating the configuration of a power application optimization system according to an embodiment of the present invention.

FIG. 14 is a flowchart illustrating the procedure of the high-frequency power application optimization method according to an embodiment of the present invention.

### DETAILED DESCRIPTION

An exemplary embodiment of the present invention will be described with reference to the accompanying drawings, and an object and the configuration, and the features of the present invention will be understood well through the detailed description.

The exemplary embodiment described above is only to describe exemplary embodiment of the present invention and is not limited to the exemplary embodiment, and various modifications and variations are possible by those skilled in the art within the spirit and claims of the present invention, and it will be said that the modifications and variations fall within the scope of the technical rights of the present invention.

Hereinafter, embodiments of the present invention entitled “System and Method for Optimizing High-Frequency Power Application Based on Electric Field Direction” will be described with reference to the accompanying drawings.

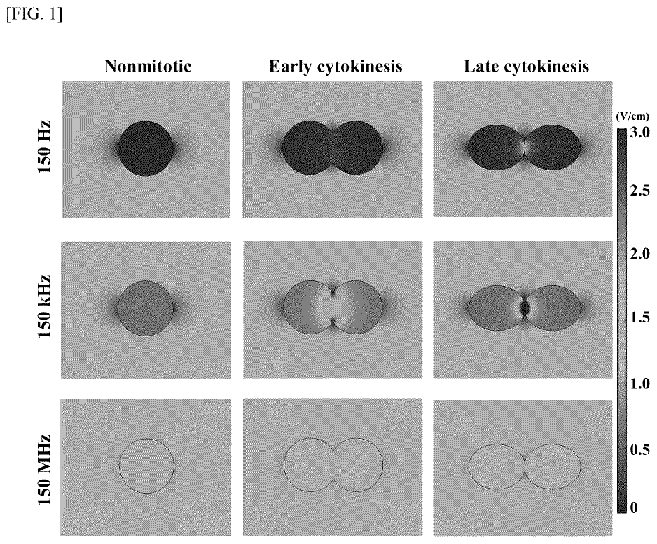

FIG. 1 illustrates the distribution of electric field intensity applied inside a tumor cell depending on the cell division state—nonmitotic, early stage of cytokinesis, and late stage of cytokinesis—and the frequency of the applied alternating electric field, which varies among 150 Hz, 150 kHz, and 150 MHz. As shown in the figure, when a 150 kHz AC electric field is applied during the late stage of cytokinesis (third column, second row), the highest electric field intensity is observed inside the dividing cell.

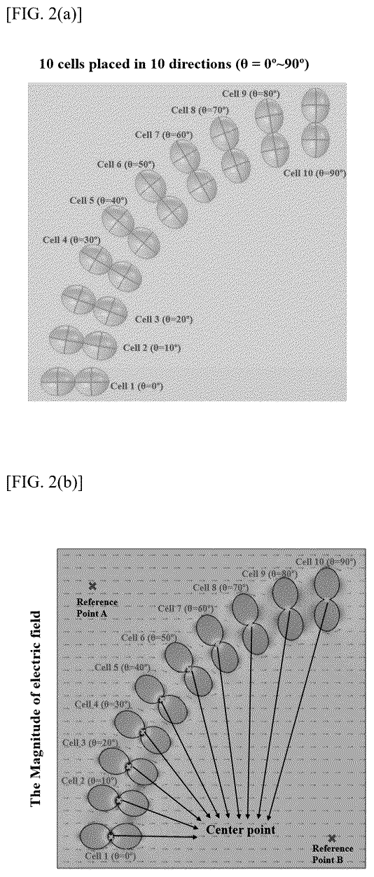

FIG. 2 shows variations in electric field intensity formed inside cells depending on the angle between the mitotic axis and the direction of the applied electric field, using a model that mimics real cells with mitotic axes oriented at various angles.

In FIG. 2 a), cells in the late stage of cytokinesis are arranged such that the angle between their mitotic axes and the direction of the applied field varies from 0° to 90° in 10° increments. A 150 kHz AC electric field is then applied. FIG. 2(b) shows the resulting distribution of electric field intensity within the dividing cells. FIG. 2(c) is a graph illustrating the magnitude of electric field intensity inside the cells as a function of the angle between the mitotic axis and the direction of the applied electric field. The maximum electric field intensity of 5.5 V/cm is observed when the angle is 0° or 180°, indicating a parallel arrangement. The field intensity decreases as the angle approaches 90°, where the directions are perpendicular.

An angle of 0° between the mitotic axis and the electric field direction indicates that the two directions are aligned in parallel, while an angle of 90° indicates they are arranged orthogonally.

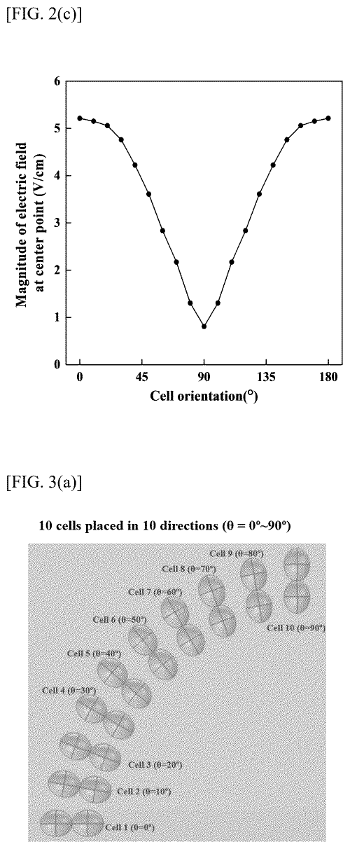

FIG. 3 illustrates the variation in dielectrophoretic force measured inside the cells, which simulates real tumor cells with mitotic axes placed at various angles. The force is analyzed as a function of the angle between the mitotic axis and the applied electric field direction.

FIG. 3(a) depicts the same arrangement as in FIG. 2(a), with a 150 kHz AC electric field applied. FIG. 3(b) shows the distribution of dielectrophoretic forces inside the dividing cells after field application. The magnitude of the dielectrophoretic force is calculated according to Equation 1 shown below.

### [Equation 1]

The magnitude of the dielectrophoretic force F applied to the cell was calculated using the following equation:

$$F = 2 π r 3 {ε}_{m} R c [ C ( ω ) ] ∇ {\vert E \vert}^{2}$$

where:
F: Dielectrophoretic force applied to the cell.
r: Radius of the cell
εₘ: dielectric constant of the cytoplasm
Rc[C(ω)]: Clausiusmossotti factor
∇|E|²: Gradient of the squared electric field magnitude

Lastly, FIG. 3(c) is a graph illustrating the magnitude of the dielectrophoretic force applied inside the cell as a function of the angle between the mitotic axis and the direction of the applied electric field. Similar to the electric field intensity shown in FIG. 2, the dielectrophoretic force reached its maximum value of 3.31310⁻¹³ N when the angle was 0° or 180° (i.e., when the directions were nearly parallel), and decreased as the angle approached 90°.

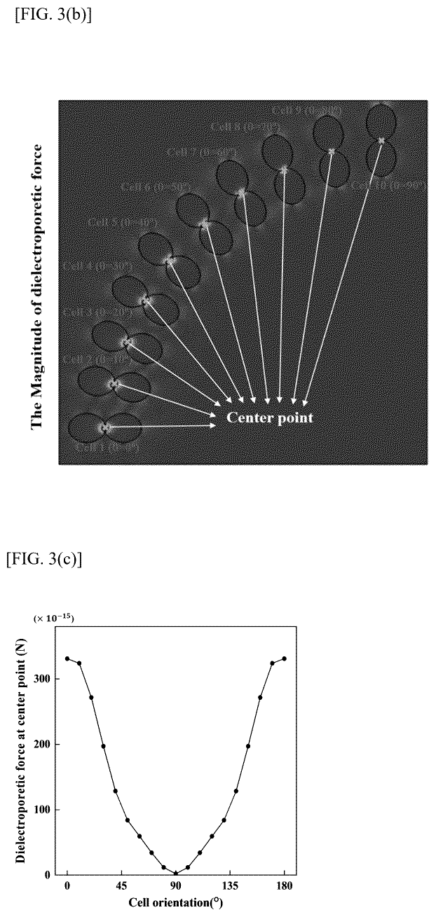

FIG. 4 illustrates the variation in the magnitude of dielectrophoretic force measured inside cells depending on the orientation of the cell and the total number of electric field directions applied, ranging from one to four.

FIG. 4(a) shows the different configurations of electric field directions applied to dividing cells. From left to right, the diagram presents: (i) a single-directional field (number of directions=1), (ii) a two-directional field applied at a 90° angle (number of directions=2), (iii) a three-directional field applied at 60° intervals (number of directions =3), and (iv) a four-directional field applied at 45° intervals (number of directions=4). Each configuration also shows the placement of the cell's mitotic axis.

FIG. 4(b) displays the measured magnitude of dielectrophoretic force inside cells with mitotic axes oriented from 0° to 180° in 10° increments, as defined in FIG. 3 (a). When two or more electric field directions are applied, it is assumed that each direction is applied for an equal amount of time. The graph indicates the maximum dielectrophoretic force among the applied directions for each given orientation of the dividing cell.

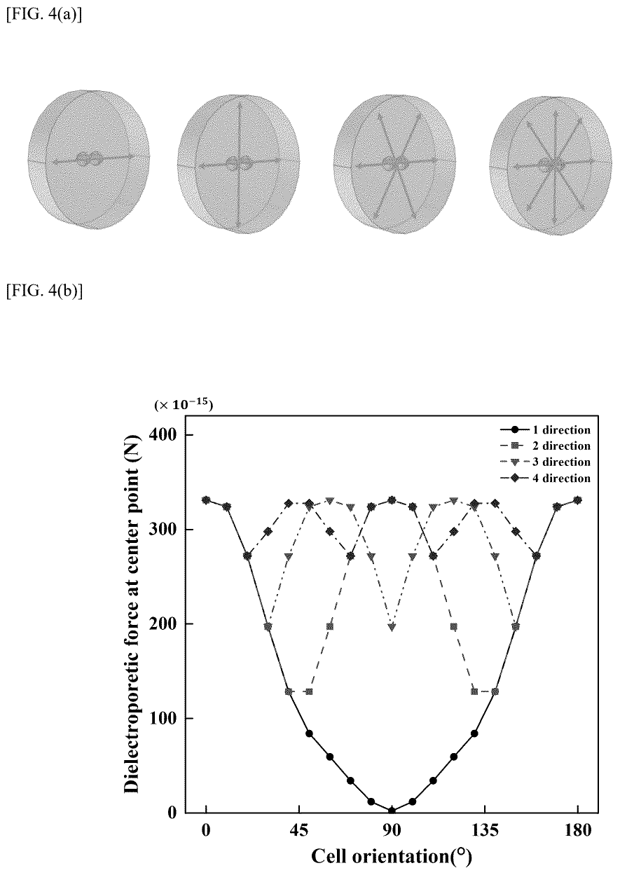

FIG. 5 presents a graph that plots the average value of the dielectrophoretic force across the 0° to 180° range for each graph shown in FIG. 4(b). These average values are expressed as relative values, normalized to 1.0 for the single-direction field case. As the number of electric field directions increases, the average magnitude of the dielectrophoretic force applied to cells also increases.

This observation suggests that applying electric fields in multiple directions enhances the overall dielectrophoretic effect on dividing cells. If the ratio of the average force generated by two, three, or four directions to that of a single direction is defined as the “total direction factor,” this factor can be calculated according to the following Equation 2.

### [Equation2]

The total direction factor is defined as the ratio of the average dielectrophoretic force when electric fields are applied in multiple directions to the average force when the field is applied in a single direction, and is calculated using the following formula:

$$Total direction factor = Average dielectrophoretic force when electric fields are applied in multiple directions / Average force when the field is applied in a single direction$$

Based on the results shown in FIG. 5, the total direction factors calculated using Equation 2 for electric fields applied in two, three, and four directions were found to be 1.62, 1.90, and 2.03, respectively. These results suggest that a higher total direction factor correlates with a greater inhibitory effect on cell proliferation caused by the electric field.

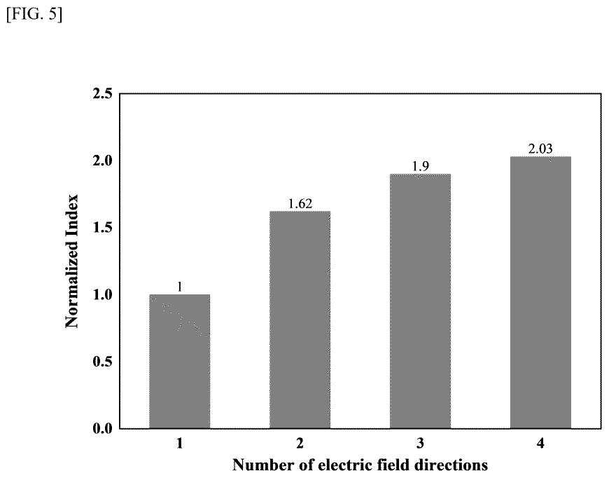

In practical scenarios where two or more electric field directions are applied, the application time for each direction may differ. Therefore, the influence of time distribution across the directions must also be taken into account. As an example, FIG. 6 illustrates the variation in the magnitude of dielectrophoretic force measured inside the cell when the electric field is applied in two directions, and the application time differs between the directions.

In FIG. 6(b), direction “A” is considered the horizontal direction and direction “B” the vertical direction. The graph presents the following three cases:
1. The electric field is applied for 4 hours in direction A only (horizontal),
2. The electric field is applied for 3 hours in direction A and 1 hour in direction B,
3. The electric field is applied for 2 hours each in directions A and B.

In each case, the dielectrophoretic force was measured using the same method as in FIG. 4(b).

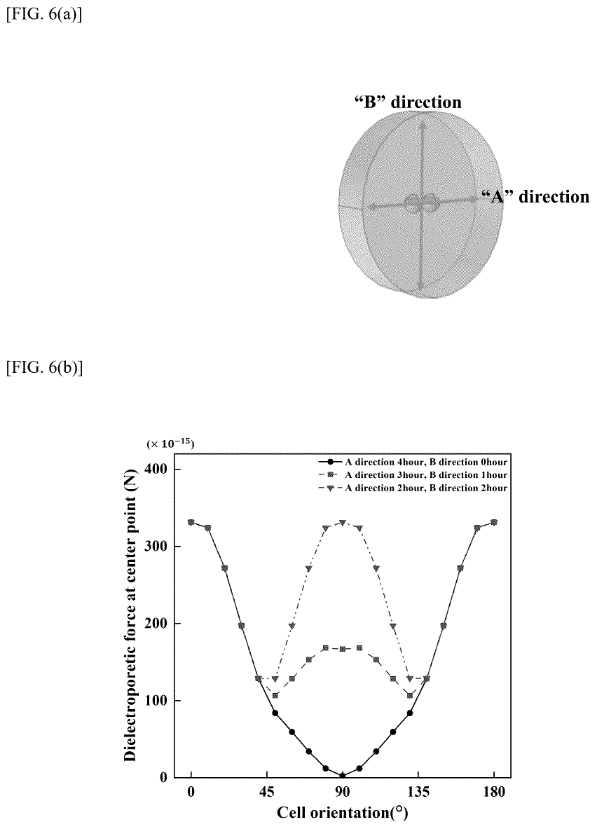

FIG. 7 shows the average dielectrophoretic force calculated over the range of 0° to 180° for each graph in FIG. 6(b), normalized to the value obtained when the electric field was applied for 4 hours in direction A only. The resulting values are plotted to reflect the effect of both the number of directions and the duration of application per direction.

For example:
In the first case (4 hours in direction A, 0 hours in direction B), since the field was applied in only one direction, the total direction factor is 1.0.
In the second case (3 hours in A, 1 hour in B), it is interpreted that for 2 hours the field was applied in one direction (A), and for the remaining 2 hours, the field was applied in two directions (A and B), assuming an equal distribution. Therefore, the time-weighted total direction factor is calculated as the average of 1.0 and 1.62, which is 1.31.
In the third case (2 hours each in A and B), since the application time was equally distributed across both directions, the total direction factor is 1.62.

Accordingly, both the original total direction factor derived in FIG. 5 and the time-weighted total direction factor reflecting the relative time per direction can be computed using the following Equation 3.

### [Equation 3]

The time-weighted total direction factor can be calculated using the following

$$Time - Weighted Total Direction Factor = {T}_{1} \cdot {D}_{1} + {T}_{2} \cdot {D}_{2} + … + TN \cdot DN / {T}_{total}$$

### Where:

Tᵢ: Duration of electric field application under the i-direction configuration
Dᵢ: Total direction factor corresponding to the number of directions used in Tᵢ
Tₜₒₜₐₗ: Total duration of electric field application

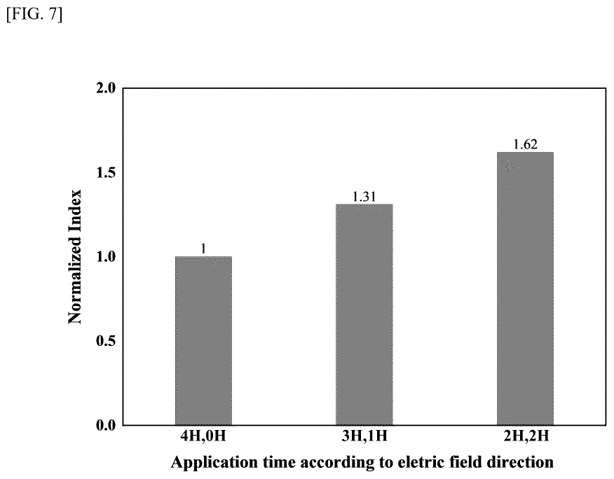

FIG. 8 illustrates the variation in the magnitude of dielectrophoretic force generated inside the cell when the electric field is applied in two directions, depending on the angle between the two directions (acute angles are considered in this invention). FIG. 8(a) shows the configurations of the angles formed between two electric field directions generated by two pairs of electrodes.

FIG. 8(b) presents the results of calculating the dielectrophoretic force inside cells depending on cell orientation, under three different angular configurations between the two directions: 30°, 60°, and 90°. The calculation was performed using the same method as described in FIG. 4(b), with the electric field applied sequentially in both directions.

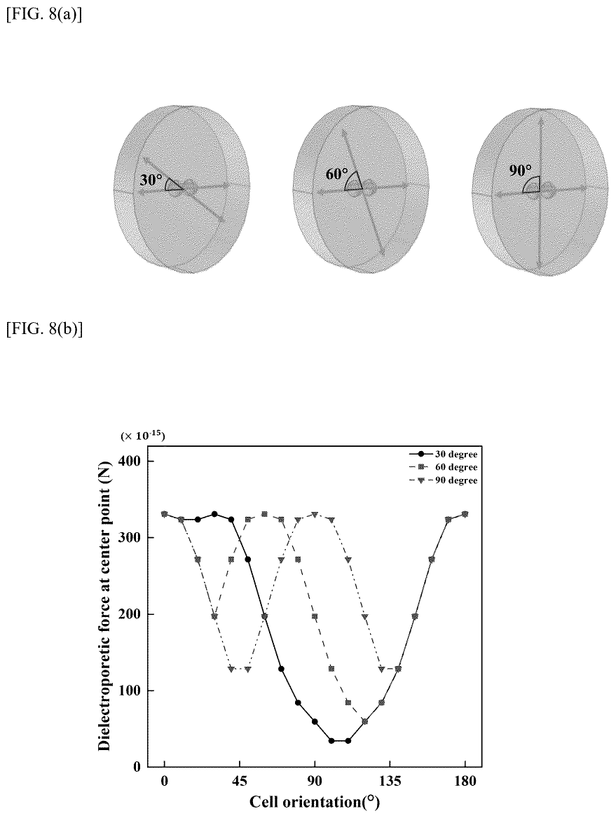

FIG. 9 is a graph in which the average dielectrophoretic force values from each of the graphs in FIG. 8(b) are calculated, and the value corresponding to the 30° configuration is normalized to 1.0. The relative average force values for the 60° and 90° configurations are then plotted accordingly.

Taken together, FIGS. 8 and 9 demonstrate that when applying electric fields using two pairs of electrodes, the magnitude of the resulting dielectrophoretic force is influenced by the angle between the field directions. Specifically, the larger the angle between the two directions, the greater the force exerted on dividing cells.

Referring to the results in FIGS. 4 to 9, it can be concluded that the magnitude of dielectrophoretic force generated inside the cell depends on the total number of electric field directions, the application time per direction, and the angle between the electric field directions. If these three factors are integrated into a single parameter, it can be defined as the Effective Total Direction Factor, which can be calculated according to the following Equation 4.

$$Effective Total Direction Factor = Time - Weighted Total Direction Factor \times Electric Field Directionality Factor \qquad [ Equation 4 ]$$

Referring again to FIGS. 4 through 9, it is evident that the magnitude of the dielectrophoretic force generated within cells depends on three primary factors: (1) the total number of electric field directions, (2) the duration of electric field application per direction, and (3) the angular relationship between the directions. These three elements are integrated into the Effective Total Direction Factor, and as this factor increases, the inhibitory effect on cell proliferation also increases. Therefore, to effectively suppress cell proliferation using high-frequency power, it is essential to optimize the Effective Total Direction Factor.

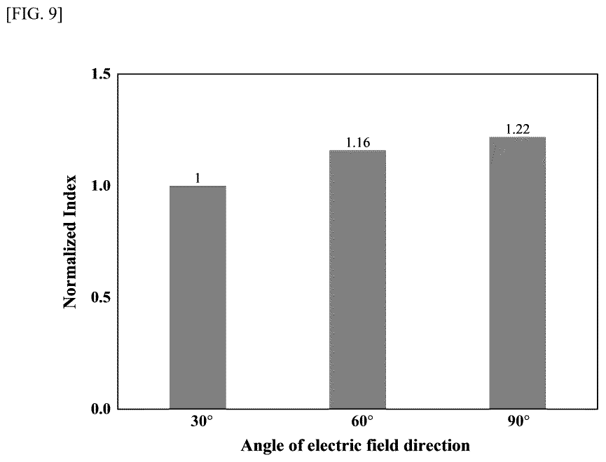

FIG. 10 illustrates the average cell viability of experimental groups relative to control groups across various cell lines, depending on the strength and duration of the externally applied electric field. The graphs in the figure are based on experimental data from brain tumor, lung cancer, gastric cancer, ovarian cancer, and cervical cancer cells.

FIG. 10(a) shows the cell viability results for external electric field intensities of 0.8 V/cm, 1.2 V/cm, and 1.6 V/cm, respectively. From this data, it can be seen that cell viability is inversely proportional to the strength of the applied electric field.

FIG. 10(b) shows the change in cell viability as a function of the electric field application time, assuming a constant field intensity. This experiment was conducted by applying an external voltage for 2, 4, 8, or 24 hours per day for three consecutive days, based on a 24-hour daily cycle. For example, when the x-axis value is “4,” it means the electric field was applied for 4 hours per day and not applied for the remaining 20 hours during each of the three days.

According to FIG. 10(b), the decrease in cell viability becomes steep as application time increases up to approximately 8 hours per day, after which the reduction rate slows significantly despite further increases in application time.

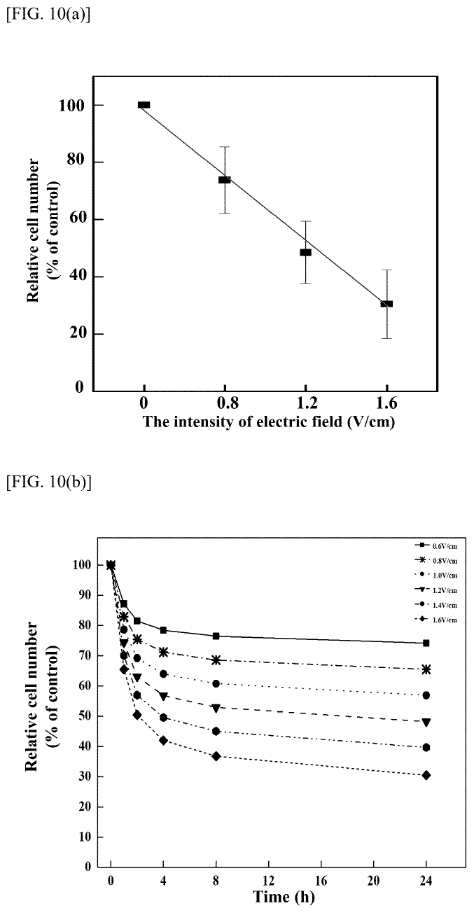

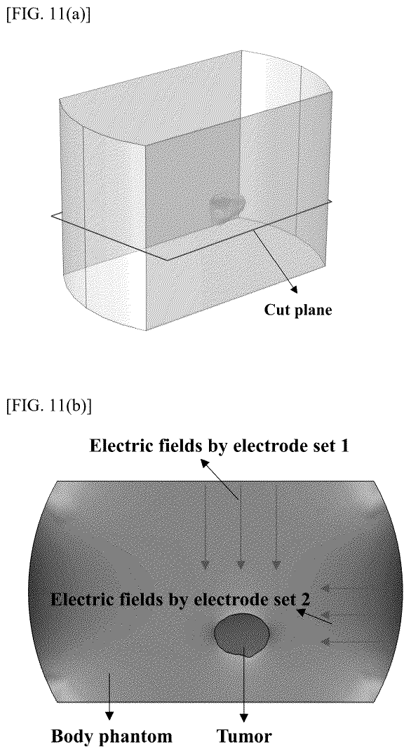

FIGS. 11 and 12 illustrate the results of cell viability measurements when electric fields are applied in two directions to a tumor located within a human body phantom. The field intensity and application time in each direction were varied.

FIG. 11(a) shows the location of the Region of Interest (ROI) within the body phantom.

FIG. 11(b) displays the distribution of electric field intensity inside the body phantom when electric fields are applied in two directions. It also provides a cross-sectional view of the phantom taken along the cutting plane indicated in FIG. 11(a).

FIG. 12(a) shows the calculated cell viability within the ROI under multiple scenarios in which electric fields of 0.6 V/cm and 1.0 V/cm were applied using two different electrode array pairs, with the application time for each array varied accordingly. The cell viability for each case was calculated using the following Equation 5.

$$Cell vitality ( \% ) = 100 - ( Cell Death Rate from Two Electrodes \times Effective Total Direction Factor ) \qquad [ Equation 5 ]$$

More specifically, in all eleven cases, the total electric field application time was fixed at 120 minutes. However, the distribution of power application time between the two electrode pairs was varied in each case. The detailed time configuration for each case is shown in Table 1 below.

TABLE 1

| | | |
|---|---|---|
| | Electric Field | Electric Field |
| case | Intensity = 0.6 V/cm | Intensity = 1.0 V/cm |

| | | | | |
|---|---|---|---|---|
| 1 | 110 | min | 10 | min |
| 2 | 100 | min | 20 | min |
| 3 | 90 | min | 30 | min |
| 4 | 80 | min | 40 | min |
| 5 | 70 | min | 50 | min |
| 6 | 60 | min | 60 | min |
| 7 | 50 | min | 70 | min |
| 8 | 40 | min | 80 | min |
| 9 | 30 | min | 90 | min |
| 10 | 20 | min | 100 | min |
| 11 | 10 | min | 110 | min |

FIG. 12(b) illustrates the calculated cell viability within the region of interest (ROI) for each case when external electric fields of 0.2 V/cm and 2.0 V/cm were applied using two pairs of electrode arrays. In each case, the electric field application time was distributed differently between the two electrode arrays.

The configuration method for applying electric fields in time segments was the same as described in FIG. 12(a). The specific time distribution for each case is provided in Table 2.

TABLE 2

| | | |
|---|---|---|
| | Electric Field | Electric Field |
| case | Intensity = 0.2 V/cm | Intensity = 2.0 V/cm |

| | | | | |
|---|---|---|---|---|
| 1 | 110 | min | 10 | min |
| 2 | 100 | min | 20 | min |
| 3 | 90 | min | 30 | min |
| 4 | 80 | min | 40 | min |
| 5 | 70 | min | 50 | min |
| 6 | 60 | min | 60 | min |
| 7 | 50 | min | 70 | min |
| 8 | 40 | min | 80 | min |
| 9 | 30 | min | 90 | min |
| 10 | 20 | min | 100 | min |
| 11 | 10 | min | 110 | min |

A combined analysis of the results from FIGS. 12(a) and 12(b) confirms that, when the number and direction of applied electric fields are fixed, the magnitude of the electric field in each direction and the application time per direction can significantly influence the cell proliferation inhibition effect in high-frequency power applications. More specifically, in FIG. 12(a), where the electric field was applied for an equal duration of 60 minutes through each electrode pair, the lowest cell viability was observed. In contrast, in FIG. 12(b), the lowest cell viability was achieved when 50 minutes of power was applied using the electrode pair delivering 0.2 V/cm and 70 minutes using the pair delivering 2.0 V/cm.

These results demonstrate that, when electric fields are applied in multiple directions using multiple electrode pairs, the cell inhibition effect can be optimized by independently adjusting both the electric field intensity and the application duration for each direction. This suggests the need for a new customized optimization approach, rather than the conventional method of applying uniform intensity and time across all directions.

From the combined results of FIGS. 4 through 12, it is evident that the inhibitory effect on cell division from high-frequency power application is dependent on:
the total number of electric field directions,
the orientation (or angle) of each direction,
the electric field intensity per direction, and
the application time per direction.

These four variables can be integrated into a comprehensive parameter and computed using the following Equation 6.

$$Cell Proliferation Inhibition Index = f ( n , {θ}_{i} , {E}_{i} , {t}_{i} ) \qquad [ Equation 6 ]$$

### Where:

n: Total number of electric field directions
θᵢ Angle or orientation of the i-th electric field direction
Eᵢ: Electric field intensity applied in the i-th direction
tᵢ: Duration of application in the i-th direction

n Equation 6, the electric field direction-related parameters—namely, the total number of directions, the directional angle factor, the electric field intensity factor per direction, and the application time factor per direction—can be mapped to corresponding components in a high-frequency power application system utilizing electrode pairs.

Specifically, these factors may be implemented as:
the total number of electrode pairs,
the angular relationship between electrode pairs (or the spatial placement of each pair),
the power intensity factor applied by each electrode pair, and
the power application time for each electrode pair.

These system-level parameters can be combined and computed according to the following Equation 7.

$$Cell Proliferation Inhibition Index = f ( N , {ϕ}_{i} , {P}_{i} , {T}_{i} ) \qquad [ Equation 7 ]$$

### Where:

N: Total number of electrode pairs
ϕᵢ: Angle or spatial placement of the i-th electrode pair
Pᵢ: Power intensity delivered by the i-th electrode pair
Tᵢ Power application time of the i-th electrode pair

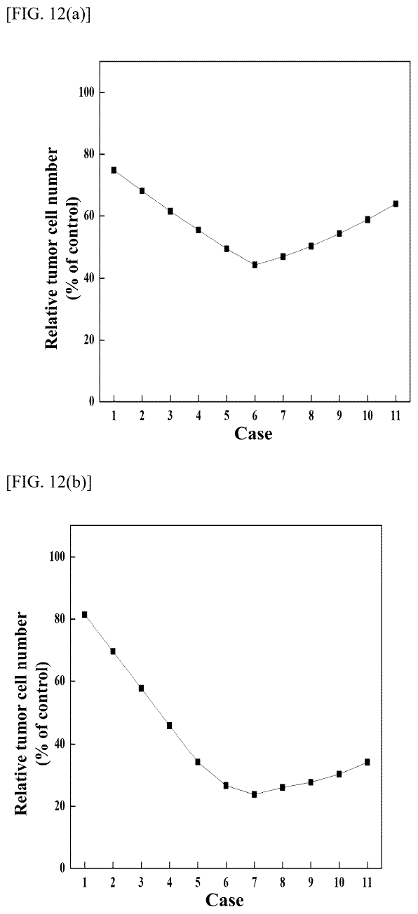

FIG. 13 is a block diagram illustrating the configuration of a power application optimization system according to an embodiment of the present invention.

The power application optimization system (100), which implements the optimization method of the present invention, includes:
an electrode pair-specific power application time setting unit (102) configured to determine the duration of power application for each electrode pair (110);
an electrode pair-specific power intensity setting unit (104) configured to determine the power intensity to be applied to each electrode pair (110); and
a power application sequence setting unit (106) configured to determine the order in which power is applied to the respective electrode pairs (110).

In addition, a switching unit (108) is configured to sequentially select and activate each electrode pair (110) for power application. Based on the switching conditions, the system determines the position of the electrode pairs (110) to which power will be applied, as well as the power intensity, application time, and application sequence for each pair.

When applying power through multiple electrode pairs (110), the power application times for each pair may be configured to be non-overlapping and executed sequentially in a repetitive manner.

When the electric field angle is varied during application, the default configuration of the power application time for each electrode pair (110) may be set to apply equal durations in a repeated, sequential fashion.

Each electrode pair (110) intended for application to a body phantom, as shown in FIG. 11, consists of individual electrodes. Each electrode may be a single electrode or a combination of two or more electrodes. When two electrodes are used, they may be placed on the front and rear sides of the target object in a symmetric arrangement. Furthermore, each electrode in an electrode pair (110) may be configured as an electrode array composed of multiple individual electrodes, for example, in a grid-like pattern with uniform spacing from adjacent electrodes in both vertical and horizontal directions.

Some individual electrodes comprising a given electrode pair (110) may also be shared with other electrode pairs (110).

As a basic configuration, the electrode pairs (110) may be spatially arranged so that the angular intervals between them are uniform.

The switching unit (108) controls the application of alternating voltage in the range of 10 to 1000 kHz to the three-dimensional subject.

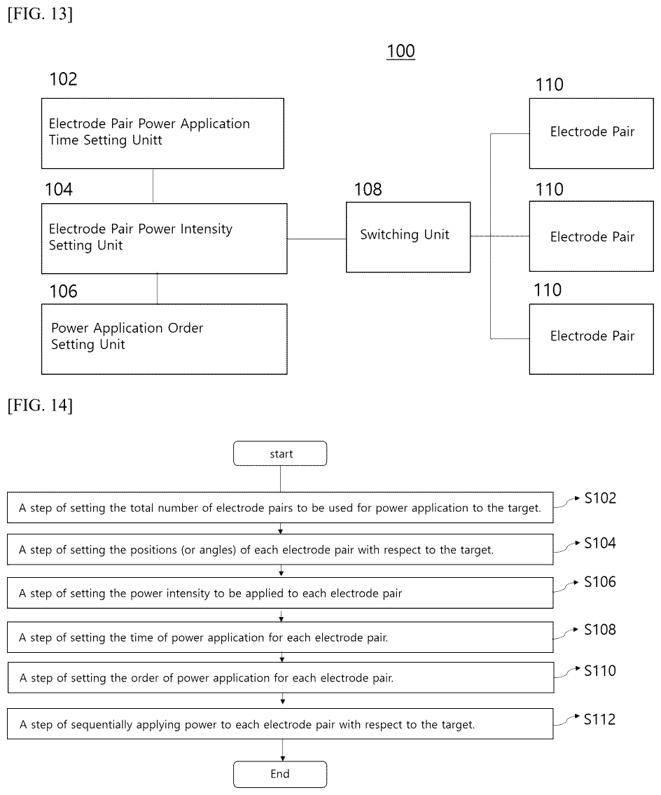

FIG. 14 is a flowchart illustrating a high-frequency power application optimization method according to an embodiment of the present invention.
1. First, the switching unit (108) sets the total number of electrode pairs (110) to be used for power application to the three-dimensional subject. (S102)
2. The switching unit (108) then sets the position or angle of each electrode pair (110) for use in the power application. (S104)
3. The electrode pair-specific power intensity setting unit (104) sets the power intensity to be applied to each electrode pair (110). (S106)
4. The electrode pair-specific power application time setting unit (102) sets the application time for each electrode pair (110). (S108)
5. The power application sequence setting unit (106) sets the application sequence for the electrode pairs (110). (S110)
6. Finally, the switching unit (108) sequentially applies power to the three-dimensional subject through the respective electrode pairs (110). (S112)

What is claimed is:

1. A high-frequency power application optimization system comprising a plurality of electrode pairs (110) configured to apply electric fields in two or more directions to a region of interest (ROI) in a three-dimensional subject, the system comprising:
a power application time setting unit (102) configured to determine the power application time for each of the electrode pairs (110);
a power intensity setting unit (104) configured to determine the power intensity applied to each of the electrode pairs (110); and
a power application sequence setting unit (106) configured to determine the order of power application to each of the electrode pairs (110);
wherein the system is configured to set the total number of electrode pairs to be used, the position of each electrode pair, the power intensity of each electrode pair, the power application time of each electrode pair, and the application sequence, so as to maximize inhibition of cell division within the region of interest.

2. The system of claim 1, wherein the power application times of the electrode pairs (100) are configured such that the application periods do not overlap and are sequentially repeated for each of the electrode pairs.

3. The system of claim 1, wherein the default configuration of power application time for each of the electrode pairs (100) is to apply equal durations in a repeated sequential manner.

4. The system of claim 1, wherein each electrode constituting the electrode pair (100) comprises one or more individual electrodes.

5. The system of claim 1, wherein each electrode constituting the electrode pair (100) is configured as an electrode array formed by a set of individual electrodes.

6. The system of claim 1, wherein a portion of individual electrodes constituting a given electrode pair (110) is shared with another electrode pair (110).

7. The system of claim 1, wherein the default positioning of the electrode pairs (100) is such that the angular intervals between adjacent electrode pairs are uniform.

8. The system of claim 2, further comprising a switching unit (108) configured to sequentially select the electrode pairs (110) for power application.

9. The system of claim 1, wherein the high-frequency power is controlled to apply an alternating voltage in the range of 10 to 1000 kHz to the three-dimensional subject.

10. A high-frequency power application optimization method for applying electric fields in two or more directions to a region of interest (ROI) in a three-dimensional subject using the high-frequency power application optimization system of claim 1, the method comprising:
setting a total number of electrode pairs (110) to be used for power application to the three-dimensional subject;
setting positions or angles of the electrode pairs (110);
setting, by the power intensity setting unit (104), the power intensity to be applied to each of the electrode pairs (110);
setting, by the power application time setting unit (102), the power application time for each of the electrode pairs (110);
setting, by the power application sequence setting unit (106), the sequence of power application for each of the electrode pairs (110); and
sequentially applying power to the three-dimensional subject through the respective electrode pairs (110);
wherein the total number of electrode pairs, the position of each electrode pair, the power intensity, the power application time, and the application sequence are configured to maximize inhibition of cell division in the region of interest.
